(19) United States
(12) Patent Application Publication
(10) Pub. No.: US 2025/0375365 A1
(43) Pub. Date: Dec. 11, 2025

(54) MOUTH RESCUE RINSE AND METHODS OF REGULATING BLOOD PRESSURE

(71) Applicant: University of Florida Research Foundation, Inc., Gainesville, FL (US)

(72) Inventors: Joseph Katz, Gainesville, FL (US); Lina Cui, Gainesville, FL (US); Zixin Chen, Gainesville, FL (US)

(21) Appl. No.: 19/227,880

(22) Filed: Jun. 4, 2025

Related U.S. Application Data
(60) Provisional application No. 63/657,355, filed on Jun. 7, 2024.

(51) Int. Cl. A61K 8/43; A61K 8/44; A61K 8/67; A61Q 11/00

(52) U.S. Cl. CPC A61K 8/43; A61K 8/44; A61K 8/676; A61Q 11/00

(57) ABSTRACT

The present disclosure provides for mouth rescue rinse, methods of using the mouth rescue rinse, and methods of regulating (e.g., maintaining or reducing) blood pressure in a subject.

### CROSS-REFERENCE TO RELATED APPLICATIONS

This application claims the benefit of: U.S. Provisional Patent Application No. 63/657,355 filed on Jun. 7, 2024, which is incorporated herein by reference in its entirety.

### BACKGROUND

Oral Rinses are extensively used to prevent and treat caries, gingivitis, and periodontal diseases. Oral antiseptic agents, such as chlorhexidine gluconate (CHX), effectively reduced both pathogenic oral bacteria and commensal oral bacterial species involved in the production of nitric oxide (NO), an important signaling molecule that regulates blood pressure by relaxing and widening blood vessels. Commensal oral bacteria are crucial for NO synthesis, acting as key participants in the enterosalivary nitrate pathway. This pathway produces NO through the sequential reduction of dietary nitrate (NO³⁻) to nitrite (NO²⁻) and then to NO. Antiseptic mouthwash can therefore lead to an increase in blood pressure.

### SUMMARY

The present disclosure provides for mouth rescue rinse, methods of using the mouth rescue rinse, and methods of regulating (e.g., maintaining or reducing) blood pressure in a subject.

The present disclosure provides for a mouth rescue rinse, comprising: a composition mixture including a saline solution and: a first component of nitrate composition; a second component of a L-arginine; or both the first component and the second component.

The present disclosure provides for a method of regulating blood pressure, comprising: administering to a subject a mouth rescue rinse as described above and herein, wherein administering includes administering before, after, or both before and after administration of an antiseptic oral rinse to the subject, wherein the mouth rescue rinse is administered into the mouth of the subject, and removing the mouth rescue rinse after a period of time from mouth of the subject, wherein the administration of the mouth rescue rinse regulates an increase in blood pressure that would result otherwise if only the antiseptic oral rinse were administered to the subject.

### DETAILED DESCRIPTION

The present disclosure provides for mouth rescue rinse and its methods of regulating blood pressure.

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art. Such techniques are explained fully in the literature.

The following description and examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in bar or psi. Standard temperature and pressure are defined as 25° C. and 1 bar.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible. Different stereochemistry is also possible, such as products of cis or trans orientation around a carbon-carbon double bond or syn or anti addition could be both possible even if only one is drawn in an embodiment.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### GENERAL DISCUSSION

The present disclosure provides for mouth rescue rinse, methods of using the mouth rescue rinse, and methods of regulating (e.g., maintaining or reducing) blood pressure in a subject. Aspects of the present disclosure can be used with or without antiseptic oral rinse. As described herein, use antiseptic oral rinse can result in disrupting the normal amounts of bacteria in the mouth and on the tongue, which can result in an increase in blood pressure in a subject. Embodiments of the mouth rescue rinse of the present disclosure reduce or eliminate the detrimental effects (e.g., increase in systolic and/or diastolic blood pressure) of antiseptic oral rinse. In an aspect, mouth rescue rinse of the present disclosure can be given before, after, or before and after the use of antiseptic oral rinse, which can reduce or eliminate the rise in systolic and diastolic blood pressure associated with using antiseptic oral rinse. In another aspect, mouth rescue rinse of the present disclosure can be used to regulate (e.g., lower) the blood pressure in normotensive and hypertensive individuals (e.g., after about 15 minutes or more after the oral application of the mouth rescue rinse).

The present disclosure provides for mouth rescue rinse. While the application references “mouth rescue rinse”, the composition mixture can be formulated as a solution, toothpaste, a gel, a lozenge, a tablet, a spray, a gum, a film or the like, where these can be used in a similar way and timing as the mouth rescue rinse disclosed herein.

In an aspect, the present disclosure provides for mouth rescue rinse that includes a composition mixture. The composition mixture includes a saline solution (e.g., a sterile saline solution) and a first component of sodium nitrate, potassium nitrate (or other nitrate salt) and/or sodium nitrite (or other nitrite salt) composition, and/or a second component of L-arginine. In an aspect, the composition mixture consists essentially of a saline solution (e.g., a sterile saline solution) and the first component, and/or the second component. The phrase “consists essentially of” means that the composition mixture does not include other active agents found in antiseptic oral rinse but can include non-active agents such as a flavoring composition, a colorant, a buffer, a salt, and the like, but does not include fluoride, chlorhexidine gluconate (CHX), or other active components found in an oral mouth rinse or oral mouthwash. In an aspect, the first component or the second component have a greater concentration than other components of the mouth rescue rinse (e.g., only the silane solution is present in a greater amount).

In an aspect, the mouth rescue rinse can include Vitamin C. In another embodiment, the mouth rescue rinse can include artificial flavors (e.g., citric acid). In an aspect, the artificial flavors can include one or more the following flavor compounds at a concentration of about 1-4%: Strawberry Concentrate, Colorless; Raspberry, Artificial; Raspberry Concentrate; Orange Concentrate, Colorless, Natural; Cherry Concentrate; Watermelon, Artificial; Butterscotch; Maple, Artificial; Marshmallow, Artificial.

In an aspect, the mouth rescue rinse can include (or consist essentially of) the composition mixture in the following amounts per 15 mL: about 15 mL of saline solution, about 100 to 300 mg or about 200 mg of NaNO₃ (or KNO₃) salt dissolved in the saline solution, about 10 to 20 mg or about 15 mg of NaNO₂ salt (e.g., about 5 mM concentration) dissolved in the saline solution and, optionally, about 500 to 1500 mg or about 1000 mg of Vitamin C dissolved in the saline solution.

In an aspect, the mouth rescue rinse can include (or consist essentially of) the composition mixture in the following amounts per 15 mL: about 15 mL of saline solution, about 500 to 1500 mg or about 1000 mg of L-arginine (e.g. about 380 mM) dissolved in the saline solution, and optionally about 500 to 1500 mg or about 1000 mg of Vitamin C dissolved in the saline solution.

The present disclosure provides for methods of regulating blood pressure in a subject. The method includes the mouth rescue rinse to the subject before (e.g., about 10 to 20 min or about 15 min before), after (e.g., about 10 to 20 min or about 15 min after or more), or both before and after administration of an antiseptic oral rinse to the subject. The mouth rescue rinse is administered into the mouth of the subject, which can be performed by the subject or someone else (e.g., nurse). After a period of time (e.g., about 30 seconds to 90 seconds), the mouth rescue rinse can be removed (e.g. spit out or sucked out) from the mouth of the subject. The mouth rescue rinse is not swallowed by the subject. This can be performed multiple times in a row and/or multiple times a day (e.g., upon waking up and before going to bed, or after meals, and the like). In an aspect, the administration of the mouth rescue rinse reduces (e.g., by about 50 to 90%) or eliminates (e.g., about 90 to 100%) an increase in blood pressure that would result otherwise if only the antiseptic oral rinse were administered to the subject. In an aspect, the mouth rescue rinse can lower (e.g. about 3 to 10 points) the blood pressure in normotensive and hypertensive individuals.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

Oral Rinses are extensively used to prevent and treat caries, gingivitis, and periodontal diseases. Oral antiseptic mouthwash and Chlorhexidine gluconate (CHX), in particular, effectively reduced bacterial species associated with oral inflammation [1].

However, the antibacterial effect of CHX is also associated with various detrimental effects on oral health, such as an increase in oral acidic condition and, as a result, a change in saliva's composition and the synthesis of oral nitrates that have a significant role in cardiovascular control [2]. Oral, commensal bacteria on the back of the tongue bacteria play an essential role in converting dietary nitrate to nitrite and the strong vasodilator nitric oxide (NO), which, combined with endogenous NO, causes vasodilation and reduces blood pressure (BP) [3].

NO is a free radical and simple gas that can be synthesized endogenously by a family of enzymes, namely NOSs. In the NOS-dependent pathway, NO is produced from the amino acid L-arginine in the presence of oxygen by NOS, and it plays an important role in preserving vascular homeostasis.

Abolishing the microbial diversity in the oral cavity can reduce the plasma nitrite levels and alter the NO/nitrite homeostasis, which may result in elevated BP levels [3]. Some studies reported that CHX rinses have resulted in higher blood pressure values in healthy subjects than in controls. In addition, a one-week CHX course was shown to significantly impact the reduction of oral microbiome associated with Oral nitrate production [4,5].

Recently, it has been reported that the oral cavity is a space in which marked changes have been detected in NO levels. Furthermore, NO is a component of saliva and is produced by oral bacteria and released by NOS expressed in the oral mucosa [6]. This information may support the notion that local application by a mouth rinse containing nitrates or L arginine that are NO substrates may result in increased production of NO and a drop in blood pressure.

HTN is a multifactorial condition known to be affected by demographic factors such as gender, race, and age, environmental factors such as smoking, and a wide range of medical conditions such as diabetes, obesity, and periodontal disease [7]. NO is a chemical that causes vasodilatation that may lower blood pressure.

Given the widespread use of CHX, we have set out to evaluate the potential association between CHX mouth rinse and treatment with nitric oxide precursors such as inorganic nitrates or L arginine mouth rinse. We hypothesize that rinsing with nitric oxide precursor will eliminate the expected rise in systolic and diastolic blood pressure. We also want to test the hypothesis that rinsing with nitric oxide can lower the blood pressure in normotensive and hypertensive individuals 15 minutes after the oral application.

### Research Plan

The study will have 3 arms, 18 in each arm.
Arm 1. Week 1, L arginine mouth rinse of 5 min, twice daily am and pm. Week 2, nonorganic nitrates rinse of 5 min twice daily am and pm.
Arm 2. Week 1, CHX rinse of 5 min, twice daily am and pm. Week 2, L arginine rinse of 5 min twice daily am and pm, followed by CHX rinse of 5 min each time.
Arm 3. Week 1, CHX rinse of 5 min, twice daily am and pm. Week 2, nitrate rinse of 5 min twice daily am and pm, followed by CHX rinse of 5 min each time.
Materials: Sodium nitrate/nitrite rinse (per 15 mL); Base: sterile saline 15 mL; NaNO₃ or KNO₃ salt: 200 mg; NaNO₂ salt: 5 mg; Base: sterile saline 15 mL; L-arginine: 1000 mg less than 1% oral intake; Vitamin C: 1000 mg; Artificial Flavor: citric acid.

The primary outcome for this study is a measured blood pressure measurement in the clinic at enrollment and the end of the study. BP will be recorded at the clinic on enrollment, after the first week and the second week, as an average of 3 consecutive. measurement at 15-minute intervals. In addition, every subject will measure his BP with a wrist cuff BP monitor provided by the study team at his home. The measurement will occur in the morning before brushing the teeth and then a rinse for 2 minutes and then another measurement 15 minutes later. This will be repeated in the evening.

On enrollment, demographic information, medical information, BMI, and BP will be recorded and the subject will be given his mouthwash for the first week. After the first week, the BP in the clinic will be taken again and the second rinse will be given to the patient. On the 3^{rd} visit, the BP in the clinic will be recorded and the subject will hand in the filled form of home BP.
Inclusion criteria: healthy subjects aged 21-80 years normotensive or hypertensive.
Exclusion criteria: subjects with a new physician diagnosis of hypertension, patients with new anti-hypertensive medications, and patients with a recent stroke or MI (within the last 3 months).

### Statistical Analysis

Demographics and clinical variables at baseline visit will be compared among the three arms. Continuous variables will be compared using Analysis of Variance (ANOVA) and Kruskal-Wallis tests as appropriate, while Pearson's chi-squared tests and Fisher's exact tests will be used to compare categorical variables as appropriate.

Intent-to-treat analyses will be conducted to compare the primary endpoints (i.e., systolic blood pressure and diastolic blood pressure) between Arm 1 and Arm 2 as well as between Arm 1 and Arm 3. Two-sample T tests and Wilcoxon-Mann-Whitney tests will be implemented to investigate between-group differences. Moreover, primary outcomes and secondary outcomes will be compared using generalized linear regressions while adjusting for demographics and other potential confounders.

Sensitivity analysis will also be conducted based on the missingness and missing pattern.

### Power Analysis and Sample Size Determination

With equal allocation to the three arms and a drop-out rate of 20%, we intend to enroll a total of 54 participants (i.e., 18 per arm).

For systolic blood pressure, we assume changes of 3.5±1.0,-0.9±0.8, and 0.9±0.8 in the three arms, respectively. For diastolic blood pressure, we assume changes of 2.2±1.0, 1.1±0.6, and 1.1±0.6 in the three arms, respectively. A total sample size of 54 will provide at least 80% power to detect the differences, with each primary test conducted at a two-sided type I error of 0.05/4 (after Bonferroni correction).

### References

1. McGrath C, Clarkson J, Glenny A M, Walsh L J, Hua F (2023). Effectiveness of Mouthwashes in Managing Oral Diseases and Conditions: Do They Have a Role? Int Dent J. 73 Suppl 2: S69-S73
2. Bescos R, Ashworth A, Cutler C, Brookes Z L, Belfield L, Rodiles A, Casas-Agustench P, Farnham G, Liddle L, Burleigh M, White D, Easton C, Hickson M (2020). Effects of Chlorhexidine mouthwash on the oral microbiome. Sci Rep. 24;10(1): 5254.
3. (Morou-Bermúdez E, Torres-Colón J E, Bermúdez N S, Patel R P, Joshipura K J (2022). Pathways Linking Oral Bacteria, Nitric Oxide Metabolism, and Health. J Dent Res. Jun;101 (6): 623-631.
4. Kapil V, Haydar S M, Pearl V, Lundberg J O, Weitzberg E, Ahluwalia A (2013). Physiological role for nitrate-reducing oral bacteria in blood pressure control. Free Radic Biol Med. Feb;55:93-100.
5. Kapil V, Rathod K S, Khambata R S, Bahra M, Velmurugan S, Purba A, S Watson D, Barnes M R, Wade W G, Ahluwalia A (2018). Sex differences in the nitrate-nitrite-NO·pathway: Role of oral nitrate-reducing bacteria. Free Radic Biol Med. ;126:113-121.
6. Ambe K, Watanabe H, Takahashi S, Nakagawa T, Sasaki J. Production and physiological role of NO in the oral cavity. Jpn Dent Sci Rev. 2016 February;52(1):14-21
7. Fuchs F D, Whelton P K (2020). High Blood Pressure and Cardiovascular Disease. Hypertension. Feb;75(2):285-292.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A mouth rescue rinse, comprising:
a composition mixture including a saline solution and: a first component of nitrate composition; a second component of a L-arginine; or both the first component and the second component.

2. The mouth rescue rinse of claim 1, wherein the composition mixture includes the saline solution and the first component.

3. The mouth rescue rinse of claim 1, wherein the composition mixture includes the saline solution and the second component.

4. The mouth rescue rinse of claim 1, wherein the composition mixture consists essentially of the saline solution and the first component.

5. The mouth rescue rinse of claim 1, wherein the composition mixture consists essentially of the saline solution and the second component.

6. The mouth rescue rinse of claim 1, wherein the composition mixture consists essentially of the saline solution, the first component and the second component.

7. The mouth rescue rinse of claim 1, further comprising Vitamin C.

8. The mouth rescue rinse of claim 1, further comprising an artificial flavor.

9. The mouth rescue rinse of claim 1, wherein the composition mixture comprises the following per 15 mL: about 15 mL of saline solution, about 200 mg of NaNO₃ salt dissolved in the saline solution, about 15 mg of NaNO₂ salt dissolved in the saline solution and about 1000 mg of Vitamin C dissolved in the saline solution.

10. The mouth rescue rinse of claim 1, wherein the composition mixture comprises the following per 15 mL: about 15 mL of saline solution, about 1000 mg of L-arginine dissolved in the saline solution, and about 1000 mg of Vitamin C dissolved in the saline solution.

11. The mouth rescue rinse of claim 1, wherein the composition mixture consists essentially of the following per 15 mL: about 15 mL of saline solution, about 200 mg of NaNO₃ salt dissolved in the saline solution, about 15 mg of NaNO₂ salt dissolved in the saline solution and about 1000 mg of Vitamin C dissolved in the saline solution.

12. The mouth rescue rinse of claim 1, wherein the composition mixture consists essentially of the following per 15 mL: about 15 mL of saline solution, about 1000 mg of L-arginine dissolved in the saline solution, and about 1000 mg of Vitamin C dissolved in the saline solution.

13. The mouth rescue rinse of claim 1, wherein the nitrate composition is selected from a nitrate, sodium nitrite composition, or an inorganic nitrate.

14. The mouth rescue rinse of claim 1, wherein the nitrate composition is selected from a nitrate, sodium nitrite composition, or a combination thereof.

15. A method of regulating blood pressure, comprising:
administering to a subject a mouth rescue rinse as described in claim 1, wherein administering includes administering before, after, or both before and after administration of an antiseptic oral rinse to the subject, wherein the mouth rescue rinse is administered into the mouth of the subject, and
removing the mouth rescue rinse after a period of time from mouth of the subject,
wherein the administration of the mouth rescue rinse regulates an increase in blood pressure that would result otherwise if only the antiseptic oral rinse were administered to the subject.

16. The method of claim 15, wherein the antiseptic oral rinse includes chlorhexidine gluconate (CHX).

17. The method of claim 15, wherein the mouth rescue rinse is administered after use of the antiseptic oral rinse.

18. The method of claim 15, wherein the mouth rescue rinse is not swallowed by the subject.

19. The method of claim 15, wherein the mouth rescue rinse is administered about 10-20 minutes before, after, or both before and after administration of the antiseptic oral rinse to the subject.

20. The mouth of claim 15, wherein the nitrate composition is selected from a nitrate, sodium nitrite composition, or an inorganic nitrate.
